(19) United States
(12) Patent Application Publication
(10) Pub. No.: US 2022/0026451 A1
(43) Pub. Date: Jan. 27, 2022

(54) METHODS FOR MEASURING AND MONTORING A MEAT CHARACTERISTIC OF A LIVESTOCK ANIMAL FROM A SAMPLE OBTAINED FROM A NON-MEAT LOCATION OF THE LIVESTOCK

(71) Applicant: C Y O'CONNOR ERAGE VILLAGE FOUNDATION INCORPORATED, Canning Vale South (AU)

(72) Inventors: Sally Stevens LLOYD, Falcon (AU); Jose L. VALENZUELA, East Perth (AU); Roger Letts DAWKINS, Canning Vale South (AU)

(21) Appl. No.: 17/299,895

(22) PCT Filed: Dec. 5, 2019
(86) PCT No.: PCT/IB2019/001355

§ 371 Date: Jun. 4, 2021

Related U.S. Application Data
(60) Provisional application No. 62/876,250, filed on Jul. 19, 2019.

(30) Foreign Application Priority Data
Dec. 5, 2018 (AU) 2018904616

(51) Int. Cl. G01N 33/92; G01N 33/483; G06N 20/00; G02B 21/36

(52) U.S. Cl. CPC G01N 33/92; G01N 33/4833; G01N 2333/46; G02B 21/367; G06N 20/00

(57) ABSTRACT

The invention is directed to a method of livestock breeding, meat production and marketing, histology, and genetics using a biopsy sample from a non-meat location of the livestock to provide information about the meat quality of the livestock.

### CROSS REFERENCE TO RELATED APPLICATION

This application claims priority to and the benefit of Australian Application Serial No. 2018904616, filed Dec. 5, 2018, and priority to and the benefit under 35 U.S.C. § 119(e) to U.S. Provisional Patent Application Ser. No. 62/876,250 filed on Jul. 19, 2019. Each reference is incorporated herein in its entirety by reference.

### FIELD OF INVENTION

The invention is directed to a method of livestock breeding, meat production and marketing, histology, and genetics using a biopsy sample from a non-meat and/or low value meat location of the livestock to provide information about the meat high value/quality of the livestock.

### BACKGROUND

Many consumers prefer meat with moderate to high (rather than low) levels of intramuscular fat (“IMF”). The composition of IMF as represented by lower melting temperature (“Tm”) of the fat is also preferred by many consumers (Pannier, L., Gardner, G. E., Pearce, K. L., McDonagh, M., Ball, A. J., Jacob, R. H., & Pethick, D. W. (2014), Associations of sire estimated breeding values and objective meat quality measurements with sensory scores in Australian lamb. Meat Science, 96(2), 1076-1087, incorporated by reference in its entirety). Animal fats with a lower Tm contain a higher proportion of unsaturated fats. The medical consensus is that such fats are healthier to consume.

Typically, as an animal develops, initial growth is in those parts which have lower economic value (bone and vital organs), followed by a period of muscle growth (of higher economic value). The amount and type of intramuscular fat is increasingly important to cattle breeders because of the many connotations for human health. High concentrations of “healthy” monounsaturated fatty acids, such as oleic acid, are known to improve the cholesterol profiles of consumers and thereby reduce the need for expensive and sometimes detrimental statin therapy. Fortunately, the same fats are associated with consumer preference suggesting that it may be simple to reduce cardiovascular complications with appropriate breed and sire selection. As the animal approaches maturity muscle growth slows and excess energy is applied to fat deposition which may include subcutaneous fat, IMF and fat surrounding internal organs. Fat deposition is energy expensive and only IMF enhances carcass value. Most livestock involved in meat production are placed on a high energy feeding regime at some stage prior to slaughter. These feeding regimes often involve high cost components and may involve housing livestock in specialized feed-lot systems. Usually, extra cost is involved.

Unfortunately, there is no agreed approach to the quantitation of marbling. In Australia, two scoring systems, MSA MB (100 to 1200) and AUS MB (1 to 9), are scored by the naked eye. A camera system is under trial. Other systems are used in Japan and the USA. Some studies have shown that the biochemical estimation of intramuscular fat can be used. So too can the melting temperature (Tm). In addition to measuring the amount of intramuscular fat, it is necessary to distinguish between coarse seams and the preferred fine marbling. This has been done by the naked eye or by camera although, again, the results differ.

“Marbling” is a term applied to IMF in meat. For instance, “highly marbled” refers to meat with a high level of IMF. Many countries have meat grading systems in which the degree of marbling forms a component in the grading system and is reported separately. In every case the degree of marbling strongly and positively influences the price received by the producer for a beef carcass. The price premium paid for marbled meat is also influenced by the distribution of fat within muscle. More evenly distributed IMF (“fine marbling”) is preferred. Marbling with a microscore of 4 or greater is characterized by hyperplasia and hypertrophy of adipoctyes surrounding the neuromuscular bundles. Further observations have confirmed that this process is associated with the expansion of the bundle itself implying that the growth factors target more than adipocytes. This explains why the assessment of microscore is facilitated by identifying the size of the bundle as well as the replacement of perimysial collagen. Not only does this assessment require less experience in histopathology, but it can be automated with simple algorithms such as neurovascular bundle (NV) diameter x adipocyte area/amount of collagen, which approximates the intuitive process used by the skilled histopathologist.

The prior art teaches that adipocytes infiltrate between the muscle bundles but the cross-section of the entire muscle remains less than would be expected if infiltration was the only mechanism. Further examples have confirmed that the proportion myocyte/adipocyte ratio actually decreases predictably. Thus, there must be a reduction in the number and size of myocytes. As illustrated, myocytes adjacent to the advancing adipocytes actually regress, shrink and are lost. So too is perimysial collagen. The process described explains for the first time why marbling is responsible for increased juiciness and flavor but also increased tenderness. It also provides a rationale for identification of the pluripotential factors responsible for marbling and related diseases in humans including muscular dystrophy, the metabolic syndrome and type 2 diabetes.

Consumer preferences and therefore market premiums vary between geographical regions and over time. In some markets, increased marbling continues to attract higher price premiums at very high levels while there is little price differential at lower marbling levels. In other markets, premium differentials are compressed at moderate to very high marbling levels, while there are substantial price differentials in the low to moderate marbling range. In some markets IMF Tm is measured and is considered a component of assessed meat quality. Carcasses with low Tm fat attract a premium price.

Two methods of direct measurement of marbling after slaughter are currently in use commercially. In visual carcass appraisal, carcasses are cut at a standardized location and are assessed by trained personnel inspecting the cut face of the relevant muscle. The proportion of fat is visually assessed on the basis of the number and size of agglomerations of fat cells and allocated a score. Different countries have different scoring systems. Australia has two systems (MSA MB and AUS MB) while Japan has a system which scores not only the overall level but also the distribution (fineness and coarseness). Appraisers can also be assisted by comparison with photographic standards. Visual appraisals are neither particularly accurate nor reproducible. While the level of training and expertise varies between different markets and countries (partly depending on the economic value attributable to the scoring), the inherent subjectivity of the visual assessment method is a source of concern to producers, consumers and commercial intermediaries alike. In photographic image processing, scoring systems using the photographic image of the cut muscle face and image processing software have been tried with limited success and have not been widely adopted. The systems are expensive to install and currently have difficulty in distinguishing fat from connective tissue and light reflected from muscle tissue. The issue of distinguishing IMF from connective tissue is particularly problem at low marbling levels in both photographic and macroscopic visual appraisal. The microscopic nature of the most valuable, fine marbling also is a confounding factor in both photographic and macroscopic visual appraisal techniques. Chemical analysis, various spectroscopic techniques and computer enhanced imaging in various spectra have been tried or are under development (Cheng, W., Cheng, J. H., Sun, D. W., & Pu, H. (2015). Marbling Analysis for Evaluating Meat Quality: Methods and Techniques. Comprehensive Reviews in Food Science and Food Safety, 14(5), 523-535 (“Cheng et al. 2015”), incorporated by reference in its entirety), demonstrating the unsatisfactory state of the methods currently in commercial use.

All current methods of measuring IMF Tm require a physical sample of the muscle to be obtained and the IMF component extracted. (Lloyd, S. S., Dawkins, S. T., & Dawkins, R. L. (2014), A novel method of measuring the melting point of animal fats, Journal of Animal Science, incorporated by reference in its entirety (“Lloyd, 2014”)). Due to the consumer preference and price premium referred to above, livestock producers have a strong interest in increasing marbling and reducing IMF Tm. These outcomes are determined by a complex interaction of an animal's genetics and environment, including the sequential feed regime. Genetic influences are partly understood (Williamson, J. F., Steele, E. J., Lester, S., Kalai, O., Millman, J. A., Wolrige, L., Dawkins, R. L. (2011), Genomic evolution in domestic cattle: Ancestral haplotypes and healthy beef, Genomics, 97(5), 304-312 (“Williamson et al. 2011”); which is incorporated by reference in its entirety). Different feeding regimes are preferred in different regions, in part depending on the availability and price differentials, with limited scientific evidence. The interactions are complex and there are indications that there are both genetic and feedstuff inhibitors and promoters of marbling and lowered IMF Tm. Many producers believe that the sequence of feeding regimes has a significant influence on marbling and IMF Tm. Within a given population of animals receiving an identical feeding regime, there is a considerable level of variation in outcomes of marbling and intramuscular fat Tm. Even where the marbling and Tm outcomes may be similar at a particular age, there is evidence that different animals may have achieved that outcome more quickly than others.

There are currently no effective commercially available methods for measuring the level of marbling in livestock prior to slaughter. Measurement of the proportion of IMF in live animals has been attempted using ultrasound imaging. While the technique is reasonably accurate in measuring the thickness of the layer of subcutaneous fat in livestock, its use in measuring IMF in Wagyu has largely been abandoned on the basis of its inaccuracy, despite its attractiveness in terms of cost and convenience. X-Ray computed tomography is reported as showing promise in a research setting (Cheng et al, 2015), but faces obvious cost and practicality difficulties in a farm or feedlot situation.

### SUMMARY OF THE INVENTION

The present invention addresses these issues by providing a method to determine at least one fat characteristic of an animal at a meat consuming region during the lifetime of the animal. Based on test samples obtained during the life of the animal, the treatment of the animal can be adjusted or the animal can be slaughtered once it reaches an acceptable level or quality of meat. The invention relies on the relationship established by the inventors between the level of marbling in muscle tissues in one part of the animal and the level of marbling in other parts. Notably, the invention is not directed to this relationship, but instead uses the relationship to determine a treatment for the animal.

The present invention has numerous advantages over current methods. Producers that could monitor the extent and, by inference, the rate of deposition of intramuscular fat in a live animal, are able to assess the extent to which continued feeding would enhance carcass value above its cost. Further, producers are able to produce to the marbling specification of a purchaser by providing animals for slaughter as they reach the desired level of marbling and can adjust their management practices to the particular market conditions they face. Further, intensive feeding of livestock prior to slaughter presents challenges to animal welfare as well as effects on costs and environmental sustainability. With methods of the present invention, producers are able to achieve their quality objectives while minimizing adverse animal welfare and environmental effects.

Marbling is an invasive, hyperplastic and hypertrophic process of intramuscular fat development. De novo perimysial fat develops initially around neurovascular bundles and expands through the perimysium in an aggressive process (arborization). As the Tm of the intramuscular fat decreases, the invasion becomes more pervasive. As a consequence, the myofibers in contact with the adipocytes suffer atrophy/degeneration and loss indicated by changes in shape and affinity to eosin. The consequence is fine marbling.

The main contributor to intramuscular fat is perimysial fat. However, the existence of endomysia adipocytes is revealed through careful histological sectioning of muscles at consecutive levels. The level of marbling and fat desaturation of meat, such as the loin, is reflected in samples obtained from a non-meat location on the animal, such as at the base of the tail. The relationship between meat and non-meat locations allows for practical tools for in vivo testing in cattle. In vivo testing, for instance through sequential biopsies, enables meat producers to determine the performance and potential for marbling and healthy fat production of individual animals.

As referred to above, the level of marbling and intramuscular fat Tm is the outcome of a complex interaction between the genetics of a particular animal and its sequential feeding regime. Commercial livestock producers, seedstock breeders, semen and embryo suppliers, and feed manufacturers and suppliers have a strong economic interest in obtaining, at the earliest possible time, accurate information as to the performance of their products in relation to marbling and IMF Tm.

Another aspect of the invention is a novel means of assessing marbling, being histological analysis of a cross-section of a sample of muscle tissue subject to assessment. The sample is assessed for the area of adipose tissue (“AAT”), the number of adipose cells within a specific area, the size of those cells and their distribution. A micro marbling scoring (microscore) system has been created to characterize the level of marbling on the basis of the muscle tissue histology. This method not only measures the proportion of IMF within the sample and its character (fineness of marbling), but the distribution can indicate the likely further development of marbling in terms of its amount and character.

The use of histological analysis of a sample allows for a muscle biopsy of a live animal to be taken so that the measurements of marbling and IMF Tm can be made during the growth of the animal and in any case prior to slaughter. By this method, changes to these meat quality parameters can be monitored in the live animal. While it may be technically feasible to take a biopsy from a live animal at the site of carcass measurement of marbling, such a procedure may present animal welfare challenges and adversely affect carcass value through tissue scarring in prime cuts. A suitable biopsy location of the present invention is at a non-meat location on the animal, i.e., a location on the animal from which cuts of meat for direct human consumption are not typically sourced in commercial meat production. For examples, a non-meat location includes the tailhead of the livestock animal as illustrated in FIG. 1, which may, if necessary, be subject to nerve blocking and so minimize pain to the animal during the biopsy procedure. A direct relationship between marbling (as measured histologically) and IMF Tm at the tailhead with marbling as measured post-slaughter between the 10^{th} and 11^{th} intercostal has been established. Therefore, measurement at the former location is an indirect measurement of marbling at the latter location. With this information, subsequent treatment for an animal during production can be determined.

Thereafter, the sample can be analyzed for the microscore, but also the Tm. The biopsy procedure at the tailhead potentially extracts sufficient IMF to allow a Tm determination using the “flip method” as described in U.S. Pat. No. 10,359,380, which is incorporated by reference in its entirety. A direct relationship between subcutaneous fat Tm at the tailhead and subcutaneous fat Tm overlying the loin has been established. There is also a direct relationship between subcutaneous fat Tm overlying the loin and IMF Tm as measured between the 10^{th} and 11^{th} intercostal. Therefore, there is a direct relationship between subcutaneous fat Tm at the tailhead and IMF Tm in the loin. The use of histological analysis to determine the proportion of IMF and its character in a livestock carcass is a superior method to that currently in use in that it is objective, accurate and reproducible.

### BRIEF DESCRIPTION OF THE FIGURES

FIG. 1 illustrates a suitable location for a biopsy sample using an embodiment of the invention;

FIG. 2 illustrates a relationship between microscore (X axis) and MSA MB (Y axis) of the Longissimus dorsi with increasing DOF;

FIG. 3A illustrates histological sections of Longissimus dorsi of three animals with increasing days on feed (DOF) and increasing marbling, with a MSA MB 290, microscore 1.5, AAT 3.0%, DOF 109, and occasional adipocytes infiltrating some parts of the perimysium;

FIG. 3B illustrates histological sections of Longissimus dorsi of three animals with increasing DOF and increasing marbling, with a MSA MB 540, microscore 4.5, AAT 10.7%, DOF 433;

FIG. 3C illustrates histological sections of Longissimus dorsi of three animals with increasing DOF and increasing marbling, with a MSA MB 1100, microscore 10, AAT 33.0%, DOF 471;

FIG. 4A illustrates histological sections of Longissimus dorsi of with a MSA MB 290, microscore 1.5, and DOF 109;

FIG. 4B illustrates histological sections of Longissimus dorsi of with MSA MB 360, microscore 1, and DOF 109;

FIG. 5 illustrates a comparison of Tm for subcutaneous and intramuscular fat of the loin of highly marbled Wagyu carcasses (R=0.85);

FIG. 6 illustrates a comparison of microscore for short fed (350 DOF) (r=0.77);

FIG. 7 illustrates a comparison of Tm of the subcutaneous fat over the Longissimus dorsi (r=0.69);

FIG. 8A illustrates a sample of Longissimus dorsi muscle with an AAT 2.3%, and microscore 1.5;

FIG. 8B illustrates a sample of Sacrocaudalis dorsalis medialis muscle with an AAT 1.3%, and a microscore 1.5;

FIG. 8C illustrates a sample of Longissimus dorsi muscle with an AAT 19.3%, and microscore 4;

FIG. 8D illustrates a sample of Sacrocaudalis dorsalis medialis muscle, with a AAT 11.2%, and a microscore 3;

FIG. 9 illustrates a relationship between Microscore of Sacrocaudalis dorsalis medialis and MSA MB score assessed at Longissimus dorsi muscle (r=0.89);

FIG. 10 illustrates a relationship between the melting temperature (Tm) of the subcutaneous fat around the base of the tail (Ischiatic tuber fat) and at the loin (11th intercostal space fat), indicating that a biopsy of tailhead fat can predict the standard result at slaughter (r=0.89);

FIG. 11 illustrates a histological section of Longissimus dorsi illustrating the presence of some apparently individual adipocytes;

FIG. 12A illustrates a Longissimus dorsi with MSA MB 580, a microscore of 3, an AAT % of 17.1% and a DOF of 429;

FIG. 12B illustrates a sample of Sacrocaudalis dorsalis medialis of a high Wagyu content steer (88%)(wy63 ak25×13), a MSA MB of 1100, DOF of 471;

FIG. 13A-E illustrates serial sections of Longissimus dorsi muscle of a 75% Wagyu, 25% Dexter heifer, at 22 months and 443 DOF, MSA MB 920, microscore 7, with arrows that illustrate the emergence and disappearance of an individual endomysial adipocyte without apparent connection to the perimysium;

FIG. 14 illustrates a frozen histological section of Longissimus dorsi muscle of a full blood Wagyu steer, at 35 months old, DOF 616, MSA MB of 1080, microscore of 9.5 (marked fibers (*) are positive to the presence of intramyocellular lipid droplets); and

FIG. 15 illustrates a histology image an example of a calculation using a NV diameter

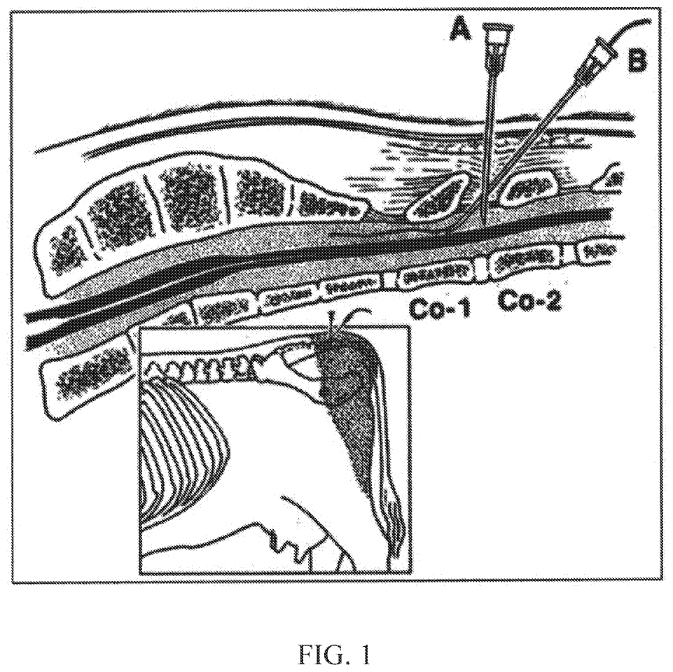

### DETAILED DESCRIPTION

The present invention provides a more accurate, objective and reproducible measure of carcass marbling, forming the basis of a more efficient market in meat at the producer, wholesale and retail level. More efficient markets will provide economic benefits for producers and consumers alike. The present invention provides, among other things, a method to treat a livestock animal based on a biopsy sample obtained at a non-consumer portion of an animal during the life of the animal. The capacity to measure and therefore monitor an individual animal's progress in the deposition of IMF and the level of IMF Tm will permit producers to make better decisions as to continued feeding of animals, environmental conditions, and time of slaughter. Decisions may be tailored to the meat quality requirements of a particular market and live animals may be sold on the basis of their current IMF and IMF Tm status as measured by the invention, to the benefit of both producer and purchaser. Notably, commercial animal producers, animal seedstock producers, commercial feed manufacturers and semen and embryo suppliers will be able to make better and earlier judgements as to the efficacy of their products and practices using the methods of the present invention. Further, genetic improvement of livestock through breeding systems will be enhanced by a more accurate and earlier system of measuring desired marbling and IMF Tm outcomes.

An aspect of the invention is a method of determining meat quality of an animal during the life of the animal. The method includes obtaining a tissue sample by biopsy from a non-consumer meat location of the animal. The biopsy can be analyzed to determine at least one fat characteristic of the tissue sample. At least one characteristic of the meat of a meat location of the animal can be determined based on the fat characteristic of the tissue sample.

The fat characteristic of the tissue sample can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana, K., Layton, C. & Bancroft, J. Bancroft's theory and Practice of Histological techniques, (Churchill Livingstone, 2012), (“Survana 2012”) incorporated by reference in its entirety). In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10^{th}-11^{th} level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm.

In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include Haematoxylin & Eosin (H&E) staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

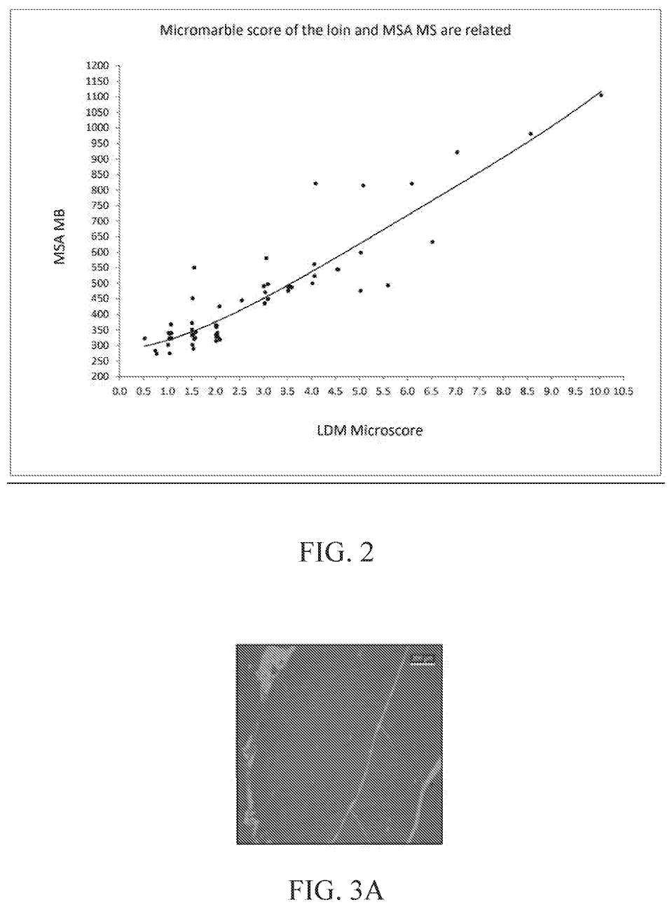

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the Meat Standards Australian Marbling (MSA MB) score, the Australian Marbling (AUS MB) score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

Once the standards score is determined from a biopsy, the treatment of the animal can be determined prior to slaughter. Suitable treatments include, slaughtering the animal, extending the feeding period of the animal, adjusting a diet of the animal, maintaining the diet of the animal, and/or modifying the amount of exercise of the animal. For example, if the microscore is not within an acceptable range for a characteristic of the animal, then the diet of the animal can be adjusted. If the animal is within the acceptable range for a characteristic of the animal, then the animal can be slaughtered. Furthermore, the microscore of the animal can be retested periodically, for example every day, every three days, every week, biweekly, bimonthly, every three weeks, every month, every two months, every quarter, three times per year, every five months, bi-yearly, every seven months, every eight months, every nine months, every ten months, every eleven months or yearly, or periodically during a range within these values. In other words, the animal can be tested at any point where a user desires additional data about the animal. When more than one sample has been obtained, the animal's progress can be tracked to determine if interventions based on previous results are altering the microscore of the animal and to determine if the intervention should be continued or if the treatment should be changed. For example, if a microscore indicated that the animal's characteristic of marbling was low, the animal's diet could be changed. If on a subsequent test for that animal, the microscore had not changed, or if the microscore became worse than a previous data point, the treatment for the animal can be changed—whether that means the animal is slaughtered or the diet is adjusted.

In some embodiments, the treatment can be determined based on a customer's input. For example, if the customer desires meat from a meat-consuming location that has significant marbling (e.g. a MSA MB score of between about 400 and about 1100), then an animal(s) can be tested before slaughtering to determine if the customer's input is met. If a customer's condition is not met, then the treatment of the animal can be modified or other animals can be tested to determine if the customer's condition has been met by the other animal.

An aspect of the invention is a method of determining a treatment of an animal. The method includes determining a standards score from a marbling score of a sample obtained from a non-meat location of an animal during the life of the animal. The treatment of the animal is determined based on the standards score. The treatment can be slaughtering the animal, adjusting an exposure condition of the animal, maintaining an exposure condition of the animal, or adjusting an exercise regimen for the animal.

The fat characteristic of the tissue sample can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana 2012. In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10^{th}-11^{th} level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm. In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include H&E staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the MSA MB score, the AUS MB score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

Once the standards score is determined from a biopsy, the treatment of the animal can be determined prior to slaughter. Suitable treatments include, slaughtering the animal, extending the feeding period of the animal, adjusting a diet of the animal, maintaining the diet of the animal, and/or modifying the amount of exercise of the animal. For example, if the microscore is not within an acceptable range for a characteristic of the animal, then the diet of the animal can be adjusted. If the animal is within the acceptable range for a characteristic of the animal, then the animal can be slaughtered. Furthermore, the microscore of the animal can be retested periodically, for example every day, every three days, every week, biweekly, bimonthly, every three weeks, every month, every two months, every quarter, three times per year, every five months, bi-yearly, every seven months, every eight months, every nine months, every ten months, every eleven months or yearly, or periodically during a range within these values. In other words, the animal can be tested at any point where a user desires additional data about the animal. When more than one sample has been obtained, the animal's progress can be tracked to determine if interventions based on previous results are altering the microscore of the animal and to determine if the intervention should be continued or if the treatment should be changed. For example, if a microscore indicated that the animal's characteristic of marbling was low, the animal's diet could be changed. If on a subsequent test for that animal, the microscore had not changed, or if the microscore became worse than a previous data point, the treatment for the animal can be changed—whether that means the animal is slaughtered or the diet is adjusted.

In some embodiments, the treatment can be determined based on a customer's input. For example, if the customer desires meat from a meat-consuming location that has significant marbling (e.g. a MSA MB score of between about 400 and about 1100), then an animal(s) can be tested before slaughtering to determine if the customer's input is met. If a customer's condition is not met, then the treatment of the animal can be modified or other animals can be tested to determine if the customer's condition has been met by the other animal.

An aspect of the invention is a method of producing an animal breeding plan for a population of at least one animal, The method includes determining a standards score or microscore for at least one animal in a population. The standards score or the microscore are ranked in an order to determine a rank in the population. Based on the rank, an animal breeding treatment can be determined.

The rank of the animal can be determined based on a desired fat characteristic of the animal. Using this information, a breeder can determine a plan for breeding two animals so that its progeny has the desired fat characteristic.

The fat characteristic of the tissue sample for each animal can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana 2012. In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10th-11th level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm. In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include H&E staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the MSA MB score, the AUS MB score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

In some embodiments, the breeding treatment can be determined based on a customer's input. For example, if the customer desires meat from a meat-consuming location that has significant marbling (e.g. a MSA MB score of between about 400 and about 1100), then two animals can be breed based on the animals microscores prior to slaughter of the animals in order to meet a desired characteristic.

An aspect of the invention is a method of determining an adjustment of an exposure condition for a population. The method includes organizing a population of animals into at least two groups. A plurality of pairs are created that include an animal from a first group of the at least two groups and a second group of the groups. An exposure condition from the first group is adjusted. Periodically, the standards score or microscore of the first group is compared to another group to determine the effect of the adjustment on the exposure condition.

In some embodiments, two groups are made. Animals with fat characteristics are divided into the two groups. One of the animals is subjected to an exposure condition and is adjusted. Periodically, the microscore and/or the standards score is measured in the two animals to determine if there is an effect on the microscore and/or standards score based on the exposure.

The fat characteristic of the tissue sample for each animal can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana 2012. In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10^{th}-11^{th} level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm. In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include H&E staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the MSA MB score, the AUS MB score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

An aspect of the invention is a method of determining changes of meat quality of an animal. The method includes obtaining a first tissue sample from the animal. The tissue sample is analyzed to determine at least one fat characteristic of the tissue sample. A microscore is assigned to the fat characteristic(s). The microscore is correlated to a characteristic of meat of an animal at the time the first sample was obtained. A second sample is obtained from the animal at a second time after the first time. The second sample is analyzed to determine at least one fat characteristic of the sample. The first and second samples are both obtained from a non-consuming meat location of the animal. The change in the fat characteristic from the first time to the second time is used to determine a treatment for the animal.

The fat characteristic of the tissue sample for each animal can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana 2012. In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10^{th}-11^{th} level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm. In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include H&E staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the MSA MB score, the AUS MB score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

Once the standards score is determined from a biopsy for the first sample, the treatment of the animal can be determined prior to slaughter. Suitable treatments include, slaughtering the animal, extending the feeding period of the animal, adjusting a diet of the animal, maintaining the diet of the animal, and/or modifying the amount of exercise of the animal. For example, if the microscore is not within an acceptable range for a characteristic of the animal, then the diet of the animal can be adjusted. If the animal is within the acceptable range for a characteristic of the animal, then the animal can be slaughtered. Furthermore, the microscore of the animal can be retested periodically, for example every day, every three days, every week, biweekly, bimonthly, every three weeks, every month, every two months, every quarter, three times per year, every five months, bi-yearly, every seven months, every eight months, every nine months, every ten months, every eleven months or yearly, or periodically during a range within these values. In other words, the animal can be tested at any point where a user desires additional data about the animal. When more than one sample has been obtained, the animal's progress can be tracked to determine if interventions based on previous results are altering the microscore of the animal and to determine if the intervention should be continued or if the treatment should be changed. For example, if a microscore indicated that the animal's characteristic of marbling was low, the animal's diet could be changed. If on a subsequent test for that animal, the microscore had not changed, or if the microscore became worse than a previous data point, the treatment for the animal can be changed—whether that means the animal is slaughtered or the diet is adjusted.

In some embodiments, the treatment can be determined based on a customer's input. For example, if the customer desires meat from a meat-consuming location that has significant marbling (e.g. a MSA MB score of between about 400 and about 1100), then an animal(s) can be tested before slaughtering to determine if the customer's input is met. If a customer's condition is not met, then the treatment of the animal can be modified or other animals can be tested to determine if the customer's condition has been met by the other animal.

An aspect of the invention is a method of harvesting an animal for meat production having meat with a pre-determined fat characteristic. The method includes obtaining a tissue sample from a non-meat location of an animal. The tissue sample is analyzed to determine at least one fat characteristic of the tissue sample. If the fat characteristic(s) is less than the pre-determined fat characteristic, feeding the animal with a high energy feed ration. After feeding the animal with a high energy feed ration, obtain a second sample of the tissue sample and analyzing the fat characteristic of the tissue sample until the determined fat characteristic of the tissue sample is at or greater than the pre-determined fat characteristic. When the determined fat characteristic of the tissue sample is at or greater than the pre-determined fat characteristic, slaughtering the animal to harvest meat with a pre-determined fat characteristic.

The fat characteristic of the tissue sample for each animal can be selected from the group consisting of an AAT, adipose cell number/area, adipose cell size, adipose cell distribution, the fat melting temperature, which can be the intramuscular fat melting temperature, and combinations thereof. The melting temperature of the subcutaneous fat sample of the animal can provide information about the intra-muscular fat melting temperature of the animal at a loin (i.e. a meat location of the animal) or other meat locations. Other characteristics of the meat at a meat consuming location of the animal can include marbling. More than one characteristic can be determined from a sample. Other characteristics can include chemical markers of “taint” flavors. Meat consuming locations include locations of cuts of meats for consumer consumption and include a forequarter cut or a hindquarter cut. Suitable forequarter cuts include for example, brisket, blade, and cube roll. Suitable hindquarter cuts include for example rump, round, tenderloin, and strip loin. Non-consumer meat locations are locations of the animal not intended for consumer consumption or not preferred for consumer consumption, and include muscle samples taken from the Longissimus dorsi at 10^{th}-11^{th} rib level, the muscle Sacrocaudalis dorsis medialis (located in the tailhead).

In some embodiments, the biopsy samples can be stored in a preservative. Suitable preservatives include formalin. In some embodiments, the concentration of the preservative can be between 5 vol. % and about 20 vol. %, in some embodiments about 10 vol. %. Methods for treatment and analysis of tissue samples are described in Survana 2012. In some embodiments, the samples can be stored between about −25° C. and about 30° C., in some embodiments about 5° C. before the samples are analyzed. Before the samples are analyzed, they can be warmed to between about 0° C. and about 50° C., in some embodiments about 25° C.

The livestock animal can be a cow, pig, sheep, chicken, bison, lamb, goat, or other consumable animal. The biopsy of a tissue sample can be taken at the tailhead of an animal. Other suitable biopsy locations include in the Ischiatic tuber region and at the 10^{th}-11^{th} level of the animal. Other suitable locations to obtain the biopsy sample can include non-consuming portions of an animal. The tissue sample can be of the Sacrocaudalis dorsis medialis muscle, a tailhead subcutaneous fat sample or combinations thereof. The animal can be subjected to a nerve blocking agent to minimize pain to the animal during the biopsy procedure. Typically, the area to be biopsied is prepared by removing or minimizing hair in the area and disinfecting the area. Tools used to obtain the biopsy include biopsy needle, which preferably has a diameter of at least 11 swg, in some embodiments between about 11 swg and about 6 swg, and a length of between about 60 mm and about 120 mm. In some embodiments, more than one sample can be collected for a single animal. In some embodiments, between 1 and 20 samples of the animal can be taken at a single time.

The biopsy tissue sample can be analyzed to determine the fat characteristic(s) of the tissue sample, and the animal. The analysis can be performed by staining the tissue sample to visualize adipose cells in the tissue sample, which can be viewed through an optical microscope. Descriptions of techniques to stain tissue samples can be found in any histology lab manual, and would therefore be understood by one skilled in the art. However, other suitable resources include Survana 2012. Staining can be performed using any suitable stain as would be understood by one having skill in the art. Examples of suitable staining materials include H&E staining, Sudan black, gomori trichrome, martis scarlet, blue trichrome, desmin, or Oil Red staining.

The fat characteristic(s) of the tissue sample from the non-consuming meat location can be determined using visual methods (traditional) or can be determined using a computer-assisted method. The method can include analyzing a digital photograph taken through an optical microscope of the stained biopsy to determine the AAT and the muscle tissue area. These areas can be distinguished by color. These areas can be determined by a user or by using a computer. In some embodiments where a computer assisted method is utilized, the computer system, which includes a processor, and programs therein can be used to calculate the AAT and/or the muscle tissue area from the biopsy. In some embodiments, the computer assisted method can use an artificial intelligence algorithm to calculate the AAT and/or the muscle tissue area from the stained biopsy.

A marbling microscore of between about 0 and 10 can be assigned to a sample based on the ratio of the adipose tissue area and the muscle tissue area. When no fat is present, the score assigned is 0. A maximum score of 10 is provided at 30% or higher fat levels. A microscore of 1 corresponds to <2%, 2 corresponds to 3%, 3 corresponds to 4%, 4 corresponds to 8%, 5 corresponds to 12%, 6 corresponds to 16%, 7 corresponds to 20%, 8 corresponds to 24%, 9 corresponds to 28%, or 10 corresponds to >30%. If necessary, the marbling microscore calculated using the present invention can be correlated to a standards score, for example the MSA MB score, the AUS MB score, or standards from other jurisdictions. FIG. 2 provides the correlation between the microscore of the present invention and the MSA MB score. Here, the r=0.91, and a plotted curve is best fit polynomial order 4. The correlation between the two measurements is most apparent as marbling increases. For MSA MB below 400 the microscore ranges from 0.5 to 2.0, suggesting that MSA MB is less discriminating.

One skilled in the art could utilize this chart to determine the correlation between the microscore of the present invention and other score standards for other jurisdictions without deviating from the present invention.

Once the standards score is determined from a biopsy for the first sample, the treatment of the animal can be determined prior to slaughter. Suitable treatments include, slaughtering the animal, extending the feeding period of the animal, adjusting a diet of the animal, maintaining the diet of the animal, and/or modifying the amount of exercise of the animal. For example, if the microscore is not within an acceptable range for a characteristic of the animal, then the diet of the animal can be adjusted. If the animal is within the acceptable range for a characteristic of the animal, then the animal can be slaughtered.

The second sample can be obtained after the first sample, which can be one hour, 12 hours, 24 hours, 3 days, 5 days, 6 days, one week, 1.5 weeks, 2 weeks, 2.5 weeks, 3 weeks, 3.5 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 52 weeks, 1.5 years, or 2 years after the first sample is obtained, or during a time period ranging between two of these time periods. If on a subsequent test for that animal, the microscore had not changed, or if the microscore became worse than a previous data point, the treatment for the animal can be changed—whether that means the animal is slaughtered or the diet is adjusted.

In some embodiments, the treatment can be determined based on a customer's input. For example, if the customer desires meat from a meat-consuming location that has significant marbling (e.g. a MSA MB score of between about 400 and about 1100), then an animal(s) can be tested before slaughtering to determine if the customer's input is met. If a customer's condition is not met, then the treatment of the animal can be modified or other animals can be tested to determine if the customer's condition has been met by the other animal.

### EXAMPLES

### Example 1

An official MSA marble score was obtained for carcasses from the abattoir determined following the procedures described in the Handbook of Australian Meat (Meat and Livestock Australia, 2005) and reported as MSA MB (100 to 1200). A microscore for microscopic assessment of the level of marbling in histological samples stained with H&E was created. The score ranges from 0 to 10, taking into consideration the proportion of adipose tissue to muscle tissue. The size of adipocytes was also noted. The scoring was done by observation of the entire histological slide with an optical microscope under a total magnification of 100×. The exercise was done between two people, one evaluator and one person providing the samples and recording the scores. Control samples were chosen for microscore level 1, 3, 5, 7 and 9 and used as blind duplicates during the scoring process. Using Leica Application Suit version 4.12 software, an objective measure was obtained. The area of adipose tissue was selected by color. Obvious connective tissue was eliminated subjectively. The percentage fat was recorded as AAT %.

### Relationship Between MSA MB Score, “Microscore” and AAT %

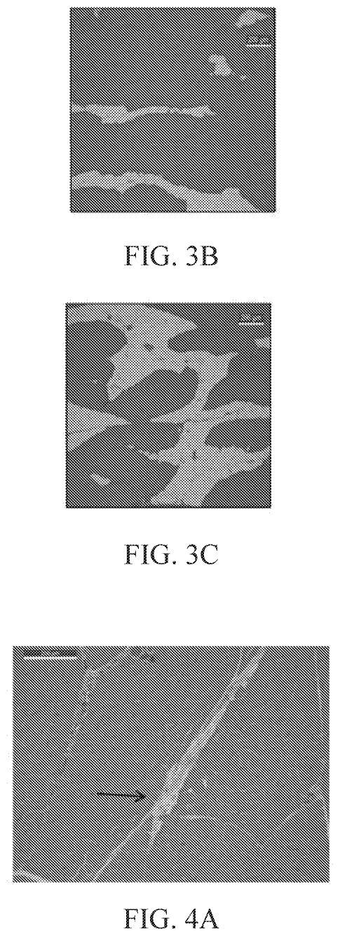

Examples of degrees of marbling of the Longissimus dorsi are illustrated in FIGS. 3A-3C, which are representative portion of a bigger sample. Microscopy of low MSA MB (FIG. 3A) illustrates a few small areas of adipocytes surrounding neurovascular bundles but also some connective tissue adjacent to adipose tissue. The two types of tissue are distinguished microscopically and only the adipose tissue is considered when allocating a “microscore” and the AAT %.

For MSA MB between 270 and 450 the AAT % was in the range 0.15% to 6.5%. As the marble score increases the area of adipose tissue increases, with more and larger collections of adipocytes. FIG. 3B illustrates histological sections of Longissimus dorsi of three animals with increasing DOF and increasing marbling, with a MSA MB 540, microscore 4.5, AAT 10.7%, DOF 433. There is more extensive invasion of the perimysium. For MSA MB between 450 to 700, the AAT % was in the range 7.5% to 25%. At high marble scores the area of adipose tissue extends as illustrated in FIG. 3C. For MSA MB above 900 the AAT % was in the range 27% to 32%. There is extreme invasion creating 434 the arborisation pattern of marbling, surrounding and separating most muscle fascicles. The adipocytes have increased in size as well as number.

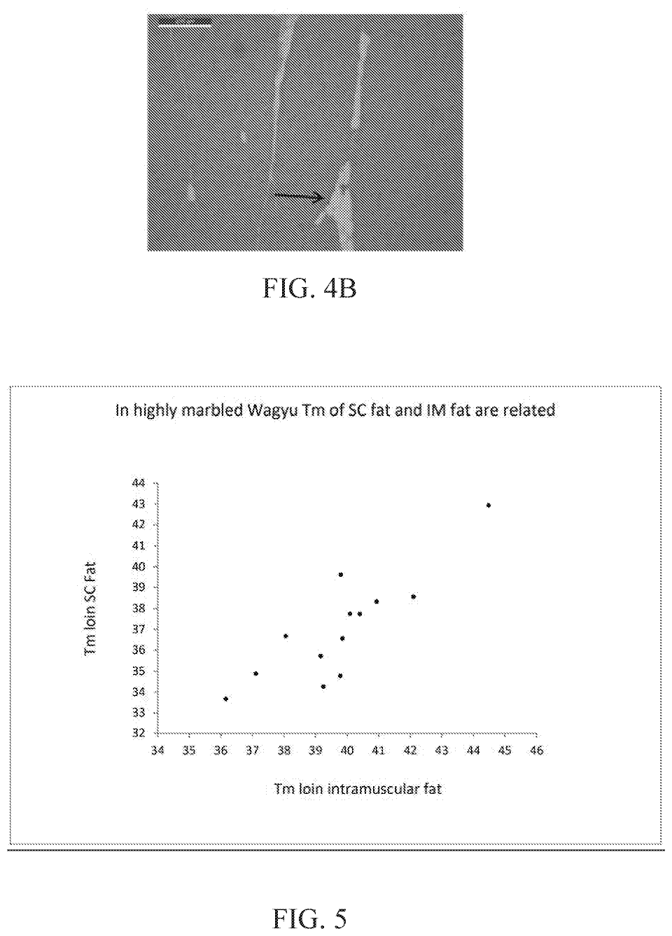

Histological examination of less marbled animals reveals connective tissue as the possible confounding factor. In some less marbled animals, there are significant areas of connective tissue that would be mistaken for fat under macroscopic examination. FIG. 4A illustrates an area of connective tissue, while FIG. 4B illustrates an area of adipose tissue. Both would appear white and be indistinguishable macroscopically. Thus, FIG. 4A illustrates histological sections of Longissimus dorsi of two animals to illustrate the risk of confusing perimysial connective tissue (FIG. 4A) and true marbling due to adipocyte invasion (FIG. 4B). In this circumstance, the microscore would give a more accurate measure of marbling than MSA MB.

### Relationship Between Subcutaneous Fat Tm and IMF Tm

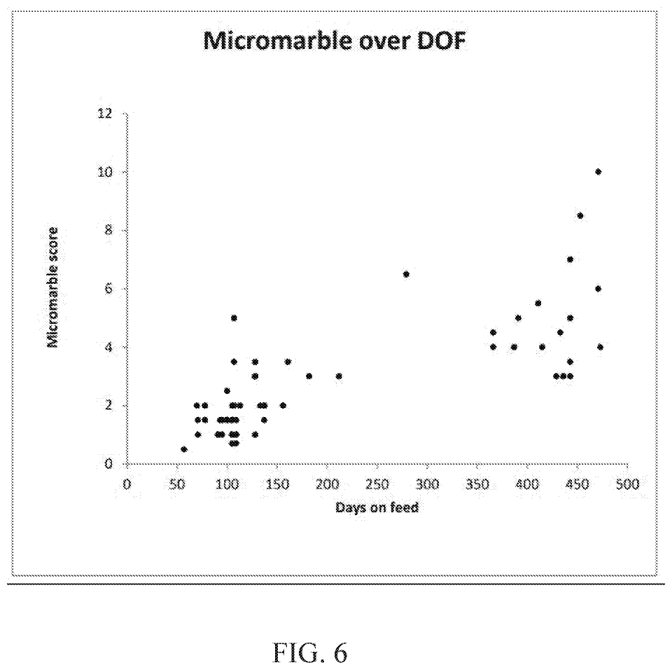

Thirteen sample sirloins (Wagyu breed) were tested to determine the IMF Tm and the Tm of the overlying subcutaneous fat. FIG. 5 plots the relationship with a correlation of 0.85 and demonstrates that subcutaneous fat can be used to monitor IMF Tm in live animals.

### Relationship Between “Microscore”, Subcutaneous Fat Tm and “Days on Feed”

Most livestock involved in meat production are placed on a high energy feeding regime prior to slaughter. The duration of this regime is referred to as “days on feed” (“DOF”). The carcasses from two groups of cattle (one group with DOF of less than 200, and one with a DOF more than 350) were tested.

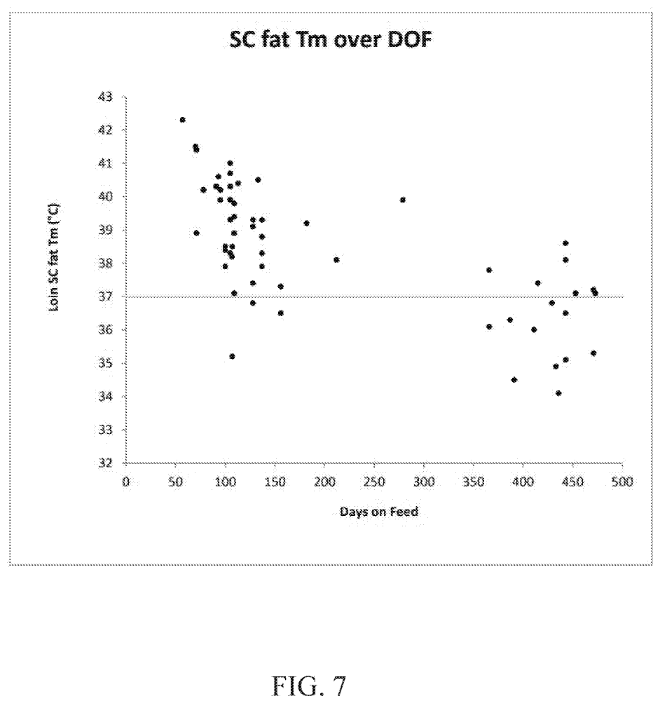

Variation of marbling with DOF is illustrated in FIG. 6. Animals with DOF less than 200 (Group 1) had a microscore below 4, while animals with DOF higher than 350 (Group 2) presented microscores from 3 to 10.

There were also qualitative differences. Group 1 had small adipocytes located in the perimysium (FIG. 3A). Group 2 had bigger adipocytes, greater variability of adipocyte size, more arborisation and a lower proportion of connective tissue in relation to adipose tissue (FIG. 3C).

Despite a clear relationship between DOF and marbling, there is substantial variation between animals at similar DOF. Animals fed between 430 and 470 days have microscores varying between 3 and 10. An example is seen by comparing FIG. 3B with FIG. 3C. Genetic factors must be responsible.

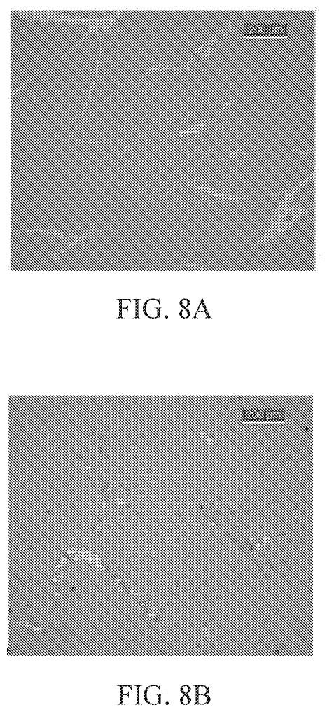

The effects of DOF on Tm of subcutaneous fat is illustrated in FIG. 7. Animals with less than 150 DOF have Tms more than 37° C. while animals with more than 350 DOF have Tms ranging from 34° C. to 38° C. There is a clear trend of decreasing Tm with increased feeding and/or age but with substantial scatter.

### Relationship Between Marbling in the Loin and Tailhead

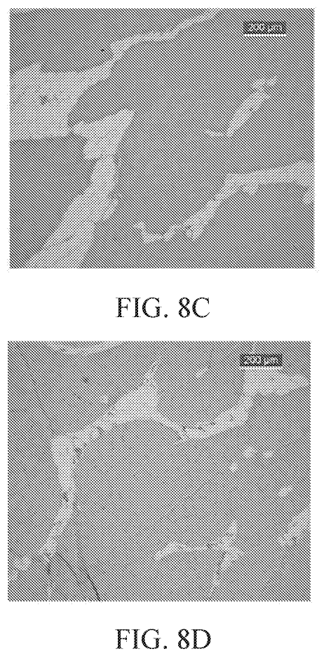

FIG. 8A-D illustrates similar intramuscular fat deposition in Longissimus dorsi muscle (FIGS. 8A and 8C) and Sacrocaudalis dorsalis medialis muscle (FIGS. 8B and 8D) at two different marbling scores, MSA MB 330 (FIGS. 8A and 8B) and MSA MB560 (FIGS. 8C and 8D). FIGS. 8A-8D were selected as a representative portion of a bigger sample. FIGS. 8A and 8B were taken from a low marble score animal, which is reflected in both SDM and LD. FIGS. 8C and 8D, from a higher marbled animal, show a proportionate increase in adipose tissue in both SDM and LD. The patterns of distribution and the abundance of adipocytes are similar between SDM and LD for both animals.

In FIG. 8A, the area of adipose tissue for the LD is higher than the SDM for both animals. This trend was found in all other animals tested. Microscores on SDM were assigned taking this into account so that microscores on LD and SDM would cover the same range (0 to 10).

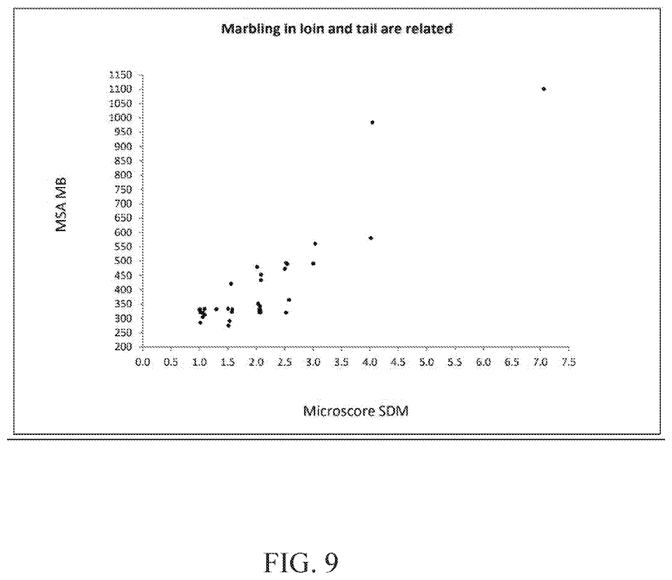

FIG. 9 illustrates the relationship between the marbling in the loin, measured by MSA MB and the microscore of the SDM. There is an excellent correlation between microscores of the SDM and the MSA MB (r=0.89).

### Relationship Between the Tm of Subcutaneous Fat Overlying the Loin and the Ischiatic Tuber

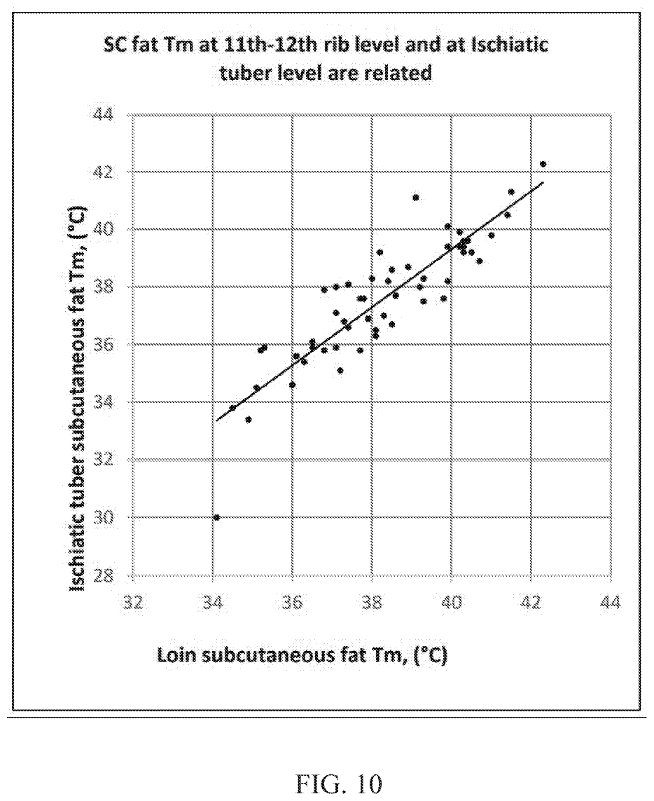

The subcutaneous fat overlying the Ischiatic tuber (IT) can also be used for in vivo sampling to monitor changes in Tm during feeding. FIG. 10 provides a relationship between the Tm of subcutaneous fat of the loin and the IT. The Tm at the IT is somewhat lower than that of the LD (r=0.89).

### Marbling

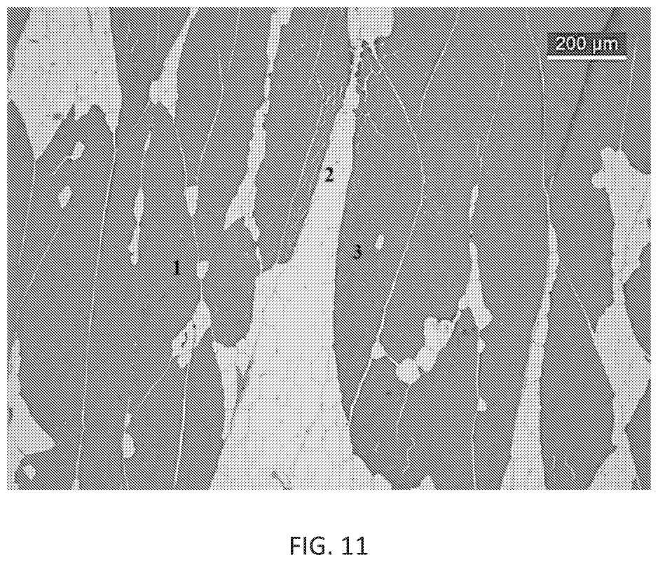

The progression of marbling, its characteristics and pattern are illustrated in FIG. 11. It is possible to identify single perimysial adipocytes, generally located close to a neurovascular bundle. There are also lines of adipocytes along the perimysium between the muscle fascicles. In the center of FIG. 11, a wedge of adipocytes has formed pushing apart the adjacent muscle fascicles. Within the wedge there is considerable variation in adipocyte size, which suggests a process of progressive arborisation, whereby adipocytes invade and separate the fascicles. Another example of advanced arborisation can be seen in FIG. 3C. At higher marbling the adipocytes are larger with more variation in 143 size (see FIG. 3A-C). As illustrated in FIG. 11, some of the adipocytes (marked as 1) are clearly within the perimysium, but others (3) appear to be isolated within the actual fasciles as might be expected if stem cells can follow the adipocyte pathway of differentiation. (2) illustrates the separation and apparent regression of myofibers as invasive progresses.

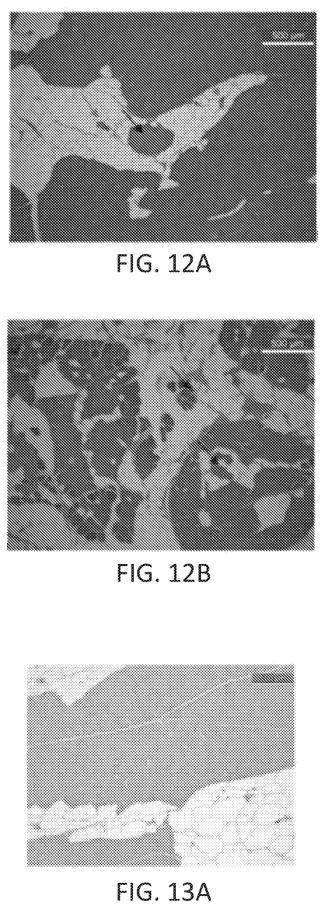

At some boundaries between adipose and muscle bundles, the perifascicular myocytes are atrophic and show changes of shape and an increased affinity to eosin. This is most often observed near the ends of branches of adipocytes (FIG. 12A-B). FIGS. 12A and 12B illustrate histological sections which illustrate the presence of neurovascular bundles marked with outer arrows in FIG. 12A and right arrow in FIG. 12B, and residual myofibers (remaining arrows). FIG. 12A illustrates a Longissimus dorsi with MSA MB 580, a microscore of 3, an AAT % of 17.1% and a DOF of 429. FIG. 12B illustrates a sample of Sacrocaudalis dorsalis medialis of a high Wagyu content steer (88%)(wy63 ak25×13), a MSA MB of 1100, DOF of 471. Islands of myocytes surrounded by adipocytes can be seen suggesting that adipose tissue invades and isolates muscle.

### Sample Use

An operator of a feedlot purchases cattle at approximately 10 months of age and provides the newly purchased cattle with a high energy feed ration. Prior to the present invention, the operator can monitor live weight gain, but otherwise has very limited means of judging the meat quality changes in the live animals so bases decisions as to whether continue feeding an animal or slaughtering it, on live weight gain. If the operator adopts the invention, on the animals entering the feedlot a biopsy is taken from the tailhead of each animal. The muscle sample from the Sacrocaudalis dorsis medalis is histologically assessed as described above and a marbling “microscore” is determined. Either the IMF Tm from the muscle sample or the subcutaneous fat Tm taken from a biopsy from fat overlying the Ischiatic Tuber is also determined as described above.

The feedlot operator repeats the biopsy procedure every three months. He is then able to more accurately assess the carcass value of each animal at each three month point, than if he used only live weight gain data. The feedlot operator can therefore determine whether further feeding of each animal is likely to be worthwhile.

The feedlot operator also has customers for beef carcasses who require product within specific meat quality parameters described in terms of marbling score and/or IMF Tm. For instance a customer may require carcasses with a MSA MB of 600 to 750 and an IMF Tm less than 38° C. The feedlot operator can select animals for slaughter on the basis of the biopsy determinations confident that they would meet those criteria, whereas if he selected animals without the information provided by the invention, a proportion of animals slaughtered would not meet the customer's requirements.

### Selection for Rapid Marbling

A farmer is producing for a market segment which requires beef with medium level of marbling (e.g. MSA MB of around 600). The less DOF required to reach that level of marbling, the lower his cost of production.

The farmer wishes to test which of his bulls is likely to produce progeny which meet the marbling objective in the lease DOF. Using the invention he is able to monitor the level of marbling in the live progeny and determine which bulls produce better outcomes. Without the invention the farmer would be required to undertake an experiment involving more animals, animals being slaughtered at less than optimum condition, and delay receiving a definitive answer to his question.

### Relationship

Postmortem samples of muscle and fat were taken from carcasses of animals harvested for routine food production. Within 2 days after birth, calves are DNA tested, confirming dam and sire and allowing later traceability of the carcasses. The calves remain with their mothers until four to six months old. At weaning the males are castrated and continue grazing kikuyu, ryegrass pasture and hay until they reach a weight of 300 kg, when they are fed pellets and ryegrass hay ad libitum. The pellets are 9 mm EasyBeef (Milne Feeds, Perth, Australia), containing lupins, barley, oats, wheat and triticale, with a nutritional composition based on dry matter of crude protein (min.) 14.5%, metabolizable energy (est.) 11.0 MJ/kg, crude fiber (max.) 20.0%, urea (max.) 1.5%, and monensin 26.6 ppm.

The calves were then divided into three groups. Group 1: Long DOF, N=17, 10 Heifers and 7 steers with Wagyu content from 50% to 96% were fed for 350 to 500 days (avg 427). Group 2: Low DOF, fed for less than 300 days (AVG 116) (N=44. 5 heifers and 39 steers, with an average MSA MB of 379). Black wagyu content was generally less than 50%. Most were 50% or more Akaushi. Group 3: Full blood and crossbreed Wagyu sirloins submitted to the 2015 Australian Wagyu Association's Branded Beef Competition from various producers around Australia (N=13. IMF 11% 79 to 54%).

After slaughter, postmortem samples of about 2-gram muscle sample was taken from the tail of each carcass. C 19 haplotypes were determined using PCR, following the method described by Williamson et al. 2017. The information resulting from this analysis was used to confirm the carcass identity and validate the data for each individual.

The fat melting temperature samples were taken at the Ischiatic tuber region and at 10^{th}-11^{th} rib level and stored at 5° C. for fat melting point analysis following the method described in U.S. Pat. No. 10,359,380 (incorporated by reference in its entirety). Muscle histology samples were taken from the muscle Longissimus dorsi, at 10^{th} and 11^{th} rib level and from Sacrocaudalis dorsalis medialis muscle for H&E and oil red O staining, and were processed as follows:
H&E: 10 mm×10 mm×3 mm samples stored in formalin 10% and then processed as 94 described in Survana 2012. The information recorded was the location of adipose 95 tissue (Perimysium, endomysium, epimysium), Adipocyte size and patterns of 96 adipocyte accumulation were noted.
Oil Red O: 3 mm×3 mm×3 mm samples, frozen in liquid nitrogen and OCT and 99 processed following the method described in Kiernan et al. 9. The aim was to visualize 100 and describe the presence of intramyocellular lipid droplets.

The official MSA grading score, provided by the abattoir and determined by naked eye following the procedures described in the Handbook of Australian Meat (Meat & Livestock Australia, Handbook of Australian Meat, 7^{th} edition (International Red Meat Manual) (2005), incorporated by reference in its entirety), was determined for each carcass. The H&E samples were read using a binocular microscope. The degree of marbling was scored from 0 to 10, taking into consideration the proportion of adipose tissue to muscle tissue. The scoring was done by two observers in turn: one blind evaluator and the other providing the samples and recording the scores. Reference samples were chosen for microscore 1, 3, 5, 7 and 9 and used as blind duplicates during the scoring process. Results were confirmed using Leica Application Suite version 4.12 software. The AAT was selected by color. Connective tissue was not included.

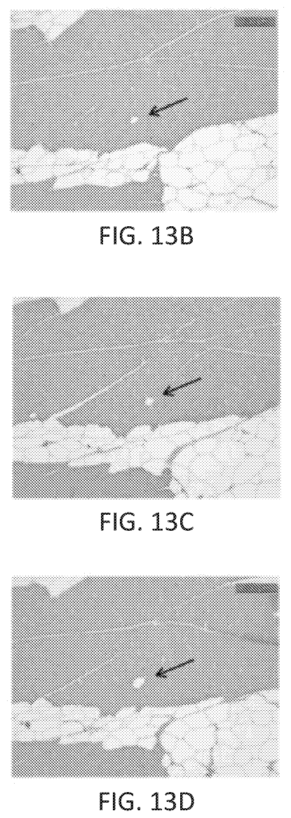

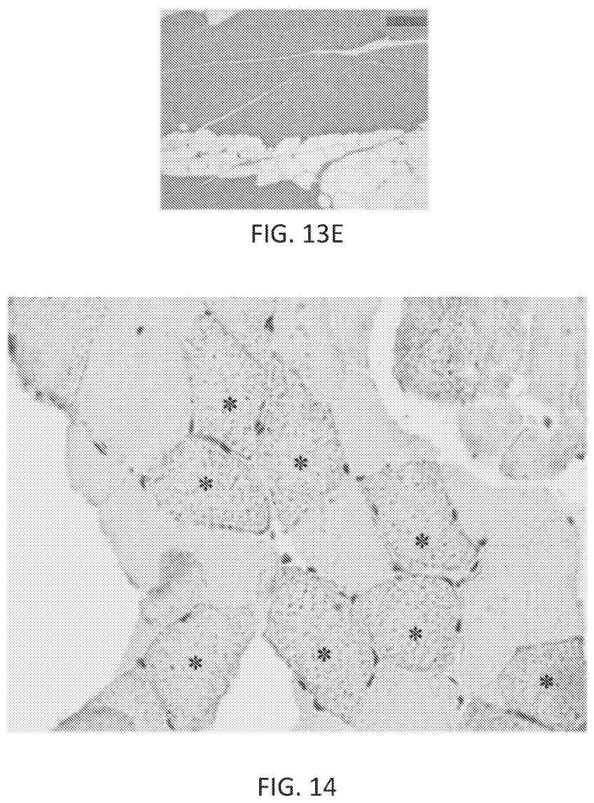

To establish their complete independence from the perimysium, serial sections of Longissimus dorsi were examined every 16 microns (FIG. 13). It is possible to see the beginning and the end of a region of endomysia adipose tissue, which confirms that this region is not connected to perimysial adipose tissue. The region was approximately 25 um wide by 50 um long, and may contain more than one adipocyte. Also noted in FIGS. 13A-E are the serration and moth-eaten appearance of the myofibers adjacent to invading adipocytes. By contrast, the borders are even where invasion has yet to occur. In one highly marbled long fed Wagyu steer, frozen sections were stained with Oil red O and the presence of intramyocellular lipid droplets was found (FIG. 14).

Histology has been widely used in human medicine to visualize, describe and understand morphological changes at a cellular level in muscle, for example in muscular dystrophies, which often involve excessive intramuscular fat deposition. The same techniques have been applied to bovine muscle to achieve similar goals, specifically to understand marbling.

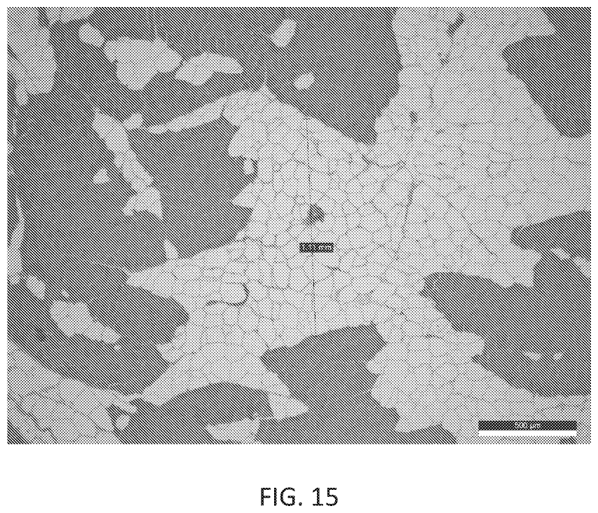

FIG. 15 illustrates a histology image an example of a calculation using a NV diameter. The microscore assigned is 8. The arteriole diameter is about 0.10 mm, the arteriole separation is about 1.21 mm, the adipose tissue width is about 1.11 mm, the collagen is about 1% and the NVB diameter*fat area/collagen is about 12.3.

Table 1 below provides the Tm for multiple samples from nine sheep.

TABLE 1

| Sample no. | Tm |
|---|---|
| 1 | 37.7 |
| 2 | 35.8 |
| 3 | 38.5 |
| 4 | 43.6 |
| 5 | 39.8 |
| 6 | 38.3 |
| 7 | 40.7 |
| 8 | 43.4 |
| 9 | 43.2 |

### Other Results

Invasive marbling is associated with concomitant decreases in Tm of fat reflecting increasing Mono Unsaturated Fatty Acids and particularly oleic acid. Thus, it is believed that the two processes are directly related. At the simplest level, lower Tm's in oils, permits greater fluidity and therefore active invasion resulting in fine marbling. This pragmatic explanation is consistent with historical practices such as the massaging of “soft” fat during the later stages of feeding of Wagyu. It also explains the fact that non-Wagyu, like Angus, retain higher Tms and are known for seam or coarse marbling. There is additional complexity. For example, there will be time dependent induction of allelic gene products encoded within Wagyu specific haplotypes such as 60.1 and 30.4. Foremost, is SREBFI which regulates the desaturase but also many other genes including many known to be involved in muscle differentiation and other potentially relevant processes. The challenge is to unravel the multitude of interacting haplotypes, genes, products and regulators especially because marbling is age and feed dependent. Experience with human muscular dystrophy teaches that current explanations for marbling are too simplistic. However, a strategy is suggested by (1) the fact that these interactions must be represented within these two haplotypes (in contrast to non-Wagyu haplotypes), (2) the opportunity to monitor activities quantitatively over time by sampling muscle and fat from the tailhead.

The microscore and MSA MB were well correlated for highly marbled beef, especially when coarse rather than fine. However, at the extremes we prefer the microscore. In carcasses with MSA MB 400 and below, the correlation is not as strong. This can be explained by the macroscopic nature of MSA MB, restricted to the naked eye visualization of white areas at least as large as 10 to 15 adipocytes. This limitation also applies to marbling assessments using a digital camera. Other types of connective tissue also appear white while the carcass is cold and are impossible to distinguish during marbling assessment. In less marbled carcasses, where the proportion of connective tissue over fat is higher, marbling can be overestimated. In contrast, a microscopic assessment of intramuscular fat is able to distinguish these details, allowing differentiation of structures and levels of marbling more completely. Therefore, the microscore is more accurate than MSA MB for the vast majority of standard, non-Wagyu carcasses.

Quantification by the naked eye can also be misleading with extreme degrees of marbling, in that it underestimates the more desired fine marbling. Some producers and some countries use an extended scale such as AUS MB 10-13. By whatever measure however, the finer the marbling the harder to quantify by the naked eye. Histological assessment will be helpful in developing measures of fineness by including the aggressiveness of the invasion and therefore the formation of residual islands of muscle and a snowflake appearance.

Marbling and Tm change with DOF but are also significantly affected by genetics. It would be useful to have methods of monitoring amounts and composition of intramuscular fat in relation to age, DOF, breed composition, suitability for breeding and animal welfare.

Worldwide, marbling is assessed in the Longissimus dorsi muscle after quartering the carcasses, but marbling occurs at different rates depending upon the muscle sampled. Obviously, biopsies of the Longissimus dorsi would not be practical. Ultrasound scanning the LD has been found unreliable for assessing marbling in long fed Wagyu. The tailhead is more accessible than the LD, more practical for biopsies under epidural anesthesia and therefore a potential site for in vivo monitoring.

The patterns of intramuscular fat deposition within LD or SDM appear to be very similar. The microscore of the SDM and the MSA MB are well correlated (r=0.89).

Therefore, the SDM is a suitable site for monitoring progression of marbling. The healthiness of beef fat, as measured by Tm, improves with feeding due to the desaturation of stearic acid into oleic acid. This process is driven primarily by the enzyme SCD (Bota 26) and regulated by SREBF1 (Bota 19). The same enzyme, regulated by is involved in desaturation of both subcutaneous and intramuscular adipose tissue. Therefore, a user can monitor changes in Tm of intramuscular fat by sampling subcutaneous fat with a simple punch biopsy, as used in humans. A substantial deposit of subcutaneous fat over the Ischiatic tuber (IT) develops in “finished” cattle and provides a practical location for sampling.

Ranges have been discussed and used within the forgoing description. One skilled in the art would understand that any sub-range within the stated range would be suitable, as would any number within the broad range, without deviating from the invention.

The foregoing description of the present invention, related to a method of treating a livestock animal by sampling a non-consuming portion of the animal, has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method of determining meat quality of an animal during life, comprising:
a. obtaining a tissue sample by biopsy from a non-meat location of the animal; and
b. analyzing the biopsy tissue sample to determine at least one fat characteristic of the tissue sample;
wherein the determined fat characteristic of the biopsy tissue sample is correlated to a fat characteristic of meat of the animal.

2. The method of claim 1, wherein the at least one fat characteristic of the tissue sample is selected from the group consisting of area of adipose tissue (AAT), adipose cell number/area, adipose cell size, adipose cell distribution, intramuscular fat melting temperature, and combinations thereof.

3. The method of claim 1, wherein the biopsy tissue sample is selected from the group consisting of a sample of the Sacrocaudalis dorsis medialis muscle and a tailhead subcutaneous fat sample.

4. The method of claim 1, wherein the step of analyzing the biopsy tissue sample to determine at least one fat characteristic of the tissue sample, comprises: staining the tissue sample to visualize adipose cells in the tissue sample and viewing through an optical microscope.

5. The method of claim 1, wherein the determination of the at least one fat characteristic is conducted using a computer-assisted method.

6. The method of claim 5, wherein the computer assisted method comprises analyzing a digital photograph taken through a microscope of the stained biopsy to determine the adipose tissue area and the muscle tissue area which can be distinguished by color.

7. The method of claim 6, wherein the computer assisted method comprises utilizing an artificial intelligence algorithm to calculate the adipose tissue area from the stained biopsy.

8. The method of claim 1, wherein said marbling microscore, between 0 and 10, and wherein said marbling microscore is determined based on said adipose tissue area and said muscle tissue area.

9. The method of claim 1, further comprising:
determining Meat Standards Australia Marbling (MSA MB) score from a chart similar to FIG. 10 and the marbling microscore; and
determining a treatment of the animal from the MSA MB score, wherein the treatment is one of: a slaughter of the animal, an adjustment of an exposure condition of the animal, or a maintenance of an animal's treatment condition.

10. The method of claim 1, wherein said chart, comprises:
an x-axis with a range of marbling microscore is from 0 to 10;
a y-axis with a range of MSAMB scores from 200 to 1200;
and a regression equation relating said range of marbling microscore to said range of MSAMB.

11. The method of claim 1, wherein the treatment comprises one of:
slaughtering the animal; or
continuing feeding regimen of the animal.

12. The method of claim 1, wherein the tissue sample is a subcutaneous fat sample of the animal; and wherein the fat characteristic is the melting temperature of the subcutaneous fat sample of the animal; and wherein the melting temperature of intra-muscular fat of the animal at a loin is correlated to the melting temperature of the subcutaneous fat sample of the animal.

13. A method of determining a treatment of an animal, comprising:
determining a Meat Standard Australia marbling score (MSA MB) from a marbling microscore; and
treating the animal by a method comprising one of:
slaughtering the animal; or
adjusting an exposure condition of the animal.

14. The method of claim 13, further comprising:
receiving a customer input expressed in terms of MSA MB;
comparing said customer input to said MSA MB to determine if said MSA MB is below, at, or above said customer input; and
wherein the treatment of the animal based on the MSA MB and the customer input comprises one of:
slaughtering if the comparison is at or above said customer input; or
adjusting the exposure condition if the MSA MB is below said customer input.

15. The method of claim 13, wherein if the treating of the animal comprises the adjusting the exposure condition of the animal;
repeating the determining of the MSA MB after a time period; and
determining an additional treatment of the animal, which comprises:
slaughtering the animal; or
adjusting the exposure condition of the animal if the MSA MB is below said customer input.

16. A method of producing an animal breeding plan for a population of at least one animal, comprising:
determining a MSA MB score for at least one animal in a population;
ranking said MSA MB scores in descending value to create a ranking of said population; and
determining the animal breeding treatment based on the ranking of said population.

17-23. (canceled)
